(19) United States
(12) Patent Application Publication
(10) Pub. No.: US 2024/0261472 A1
(43) Pub. Date: Aug. 8, 2024

(54) PRINTABLE BIOINK AND METHOD OF PRINTING A TISSUE/ORGAN MODEL OR THERAPEUTIC CONSTRUCT

(71) Applicant: President and Fellows of Harvard College, Cambridge, MA (US)

(72) Inventors: Daniel S. REYNOLDS, Cambridge, MA (US); Jennifer A. Lewis, Cambridge, MA (US)

(73) Assignee: President and Fellows of Harvard College, Cambridge, MA (US)

(21) Appl. No.: 18/567,448

(22) PCT Filed: Jun. 6, 2022
(86) PCT No.: PCT/US2022/032324

§ 371 Date: Dec. 6, 2023

Related U.S. Application Data
(60) Provisional application No. 63/208,599, filed on Jun. 9, 2021.

(51) Int. Cl. A61L 27/48; A61L 27/54; B29C 64/106; B29C 64/40; B33Y 10/00; B33Y 70/00; C09D 11/03; C09D 11/04; C09D 11/08; C09D 11/14

(52) U.S. Cl. CPC A61L 27/48; A61L 27/54; B29C 64/106; B29C 64/40; C09D 11/03; C09D 11/04; C09D 11/08; C09D 11/14; B33Y 10/00; B33Y 70/00

(57) ABSTRACT

A bioink for extrusion-based printing includes an extracellular matrix (ECM) precursor comprising an uncrosslinked polymer, and sacrificial microparticles dispersed in the ECM precursor. The sacrificial microparticles have a melting temperature above a crosslinking temperature of the uncrosslinked polymer. A method of fabricating a tissue/organ model or therapeutic construct comprises extruding a bioink comprising a first ECM precursor and first sacrificial microparticles through a nozzle moving relative to a deposition bath, and depositing an extruded filament comprising the bioink into the deposition bath as the nozzle moves. After deposition, the first ECM precursor is crosslinked to form a first ECM material, and after the crosslinking, the first sacrificial microparticles are melted to form pores in the first ECM material. The pores may have a width or diameter comparable to that of individual cells.

### FEDERALLY SPONSORED RESEARCH OR DEVELOPMENT

This invention was made with government support under CA214369 awarded by the National Institutes of Health (NIH). The government has certain rights in the invention.

### TECHNICAL FIELD

The present disclosure is related generally to extrusion-based printing and, more particularly, to a printable bioink and a method of fabricating a tissue/organ model or a therapeutic construct that may be implanted.

### BACKGROUND

In many soft tissues, the physical properties of the extracellular matrix (ECM) within the in vivo microenvironment can be described by three parameters: pore size, viscoplasticity, and degradability. Importantly, these parameters determine whether cells experience three-dimensional confinement. Confined cells may be restricted in their ability to proliferate, spread, and migrate, and therefore biomaterials that cause cellular confinement may not faithfully recapitulate the in vivo microenvironment. For example, polyethylene glycol (PEG), alginate, and hyaluronic-acid-based hydrogels, which are polymers commonly used in the field of tissue engineering, typically have nanometer-scale instead of microscale pores, predominantly elastic mechanical properties, and limited degradability. As explained below, bioinks suitable for extrusion-based bioprinting may exhibit shear-thinning behavior, which allows both flow through the nozzle and shape retention of the extruded filament upon deposition. However, this design preference may be in conflict with the additional objective that the printed ECM material permits fundamental cellular processes. Consequently, prior approaches to fabricate bioinks have been limited by compromises between printability and biocompatibility.

### BRIEF SUMMARY

A bioink for extrusion-based printing includes an extracellular matrix (ECM) precursor comprising an uncrosslinked polymer, and sacrificial microparticles dispersed in the ECM precursor. The sacrificial microparticles have a melting temperature above a crosslinking temperature of the uncrosslinked polymer.

A deposition bath for extrusion-based printing includes an extracellular matrix (ECM) precursor comprising an uncrosslinked polymer, and sacrificial microparticles dispersed in the ECM precursor. The sacrificial microparticles have a melting temperature above a crosslinking temperature of the uncrosslinked polymer.

A method of fabricating a tissue/organ model or therapeutic construct comprises extruding a bioink comprising a first extracellular matrix (ECM) precursor and first sacrificial microparticles through a nozzle moving relative to a deposition bath, and depositing an extruded filament comprising the bioink into the deposition bath as the nozzle moves. After deposition, the first ECM precursor is crosslinked to form a first ECM material, and after the crosslinking, the first sacrificial microparticles are melted to form pores in the first ECM material. The pores may have a width or diameter comparable to that of individual cells.

A method of fabricating a tissue/organ model comprises extruding a bioink through a nozzle moving relative to a deposition bath, where the deposition bath comprises a first extracellular matrix (ECM) precursor and first sacrificial microparticles. An extruded filament comprising the bioink is deposited into the deposition bath as the nozzle moves. After deposition, the first ECM precursor is crosslinked to form a first ECM material, and after the crosslinking, the first sacrificial microparticles are melted to form pores in the first ECM material. The pores may have a width or diameter comparable to that of individual cells.

### BRIEF DESCRIPTION OF THE DRAWINGS

FIG. 1 is a schematic of an exemplary extrusion-based printing process of a bioink into a deposition bath.

FIGS. 2A and 2B are schematics showing part of the extruded filament of FIG. 1 before (FIG. 2A) and after (FIG. 2B) crosslinking of the ECM precursor and melting of the sacrificial microparticles.

FIG. 3A is a microscope image of sacrificial microparticles comprising type A gelatin and chitosan, where the scale bar is 100 microns.

FIG. 3B shows the size distribution of the microparticles of FIG. 4A.

FIG. 4 shows viscosity as a function of shear rate for collagen alone, collagen with gelatin-chitosan microparticles, and gelatin-chitosan microparticles alone.

FIGS. 5A and 5B are schematics showing part of a deposition bath before (FIG. 5A) and after (FIG. 5B) crosslinking of the ECM precursor and melting of the sacrificial microparticles.

FIG. 5C shows modulus as a function of time for a bioink comprising uncrosslinked collagen with gelatin-chitosan microparticles, where the bioink is exposed to temperatures of 4° ° C., 21° C. and then 37° C.

FIGS. 6A-6F shows images of a cellular bioink including B16-F10 melanoma cells (approximately 250 million cells/mL) in phosphate-buffered saline, which is printed into either (FIGS. 6A-6C) a deposition bath of collagen and gelatin-chitosan microparticles or (FIGS. 6D-6F) collagen alone.

FIGS. 7A-7E show immunofluorescent images of cells printed within a collagen/microparticle deposition bath revealing that the cells are able to spread and are proliferative.

FIGS. 8A-8C show results from a migration assay of bone marrow-derived dendritic cells (BMDCs) embedded within a collagen/microparticle bioink or bath.

### DETAILED DESCRIPTION

Described herein is a new class of bioinks for extrusion-based bioprinting that incorporates sacrificial microparticles to provide two functions: first, to act as rheological modifiers during the printing process, and second, to function as porogens to create a microporous material upon removal after printing. The use of sacrificial microparticles as rheological modifiers broadens the biofabrication window, allowing previously unprintable or difficult-to-extrude biomaterials, such as native ECM proteins and synthetic polymers, to be printed. In addition, the sacrificial microparticles may be sized to form pores analogous in size to individual cells, thereby providing a desirable cellular environment in the printed ECM material. This new class of bioinks has the potential to expand the palette of printable biomaterials while providing a microenvironment that is conducive to key cellular processes, such as proliferation, migration, spreading, differentiation, and organoid formation.

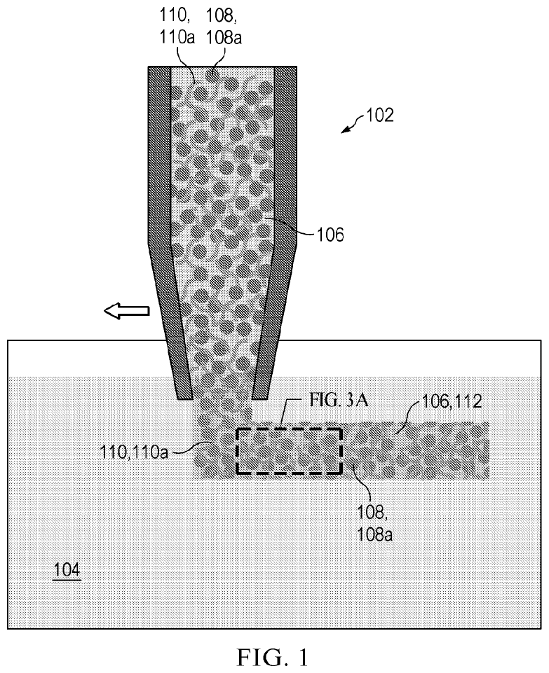

FIG. 1 shows a schematic of an exemplary extrusion-based printing process, which may sometimes be referred to as 3D printing. Ink formulations suitable for extrusion-based printing, such as the bioink 106 described in this disclosure, can be readily extruded through a nozzle 102 moving relative to a deposition bath or substrate 104. Such ink formulations and bioinks 106 may exhibit shear-thinning rheological behavior, which allows for reduced viscosity under shear forces to permit flow through a nozzle 102 and increased stiffness after extrusion to maintain the extruded shape. During extrusion-based printing, the nozzle 102 can be moved at a constant or variable speed along a desired print path while the deposition bath/substrate 104 remains stationary. Alternatively, the bath/substrate 104 may be moved while the deposition nozzle 102 remains stationary, or both the deposition nozzle 102 and the bath/substrate 104 may be moved. Descriptions of nozzle motion in this disclosure are understood to encompass all of these options.

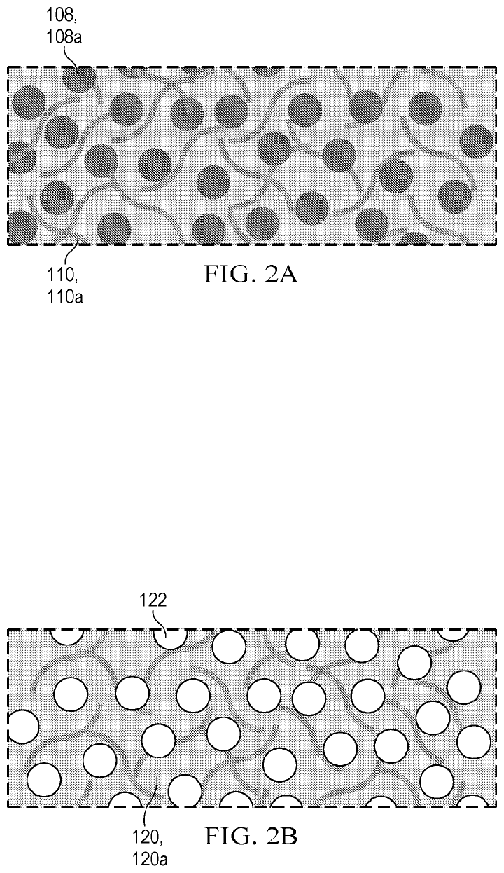

The bioink 106 may comprise an extracellular matrix (ECM) precursor 110 including an uncrosslinked polymer, and sacrificial microparticles 108 dispersed in the ECM precursor 110, as shown in FIGS. 1 and 2A. The sacrificial microparticles 108 have a melting temperature above a crosslinking temperature of the uncrosslinked polymer. Thus, after extrusion-based printing and crosslinking of the ECM precursor 110 to form an ECM material 120 comprising a crosslinked polymer, as described in more detail below, the sacrificial microparticles 108 may be heated to a temperature at or above the melting temperature. As the microparticles 108 melt, porosity 122 that can support cellular processes is introduced into the ECM material 120, as illustrated in FIG. 2B. For simplicity, the term “polymer” may be used to refer to either or both the uncrosslinked polymer of the ECM precursor 110 and the crosslinked polymer of the ECM material 120. Crosslinking may be induced by heat, light, an enzyme and/or a chemical agent, but in each scenario the crosslinking temperature of the uncrosslinked polymer may be understood to refer to the temperature at which the crosslinking reaction takes place. The polymer may comprise any natural or synthetic polymer, such as, for example, collagen, fibrinogen, reconstituted extracellular matrices, modified matrix-derived proteins (e.g., gelatin), modified glycosaminoglycans (e.g., hyaluronic acid, chondroitin sulfate), modified polysaccharides (e.g., alginate, dextran, chitosan), and functionalized polyethylene glycol. The polymer may be modified/functionalized with photocrosslinkable moieties like methacrylate, acrylate, norbornene, and/or allyl groups. Alternatively, the polymer may be modified/functionalized with dynamic covalent crosslinkers (e.g., imine and acylhydrazone) and/or host-guest interactions (e.g., adamantane and beta-cyclodextrin).

The deposition bath 104 into which the bioink 106 is deposited may comprise an ECM precursor 110, which may be the same as or different from the ECM precursor of the bioink 106. The deposition bath 104 may also or alternatively include sacrificial microparticles 108, as described below. It is also contemplated that the deposition bath 104 and/or the bioink 106 may include one or more cell types, drugs, toxins, vaccines, proteins, and/or hormones, such as growth factors, growth inhibitors, cytokines, steroids and/or morphogens. In examples where the deposition bath 104 and/or the bioink 106 include one or more cell types, the cells may be incorporated as single cells, cell clusters/aggregates, and/or organoids. It may be advantageous to encapsulate the cells (individually or in clusters) within an immunoprotective polymeric hydrogel, particularly for therapeutic applications. For example, a cell (or multiple cells) having a potential metabolic function may be encapsulated within an immunoprotective polymeric hydrogel to protect the implanted therapeutic construct from being rejected by the host. It is also contemplated that the sacrificial microparticles 108 themselves may include one or more substances, such as an enzyme (e.g., thrombin), drug, toxin, vaccine, protein, and/or hormone, as described above, which is/are released from the sacrificial microparticles 108 upon melting. Using this new extrusion-based printing approach, a tissue/organ model may be constructed.

Typically, the sacrificial microparticles 108 have a linear size (e.g., a width or diameter) in a range from about 1 μm to about 500 μm, or from about 5 μm to about 100 μm, and the linear size may also be in the range from 5 μm to about 25 μm. Upon melting, pores 122 having a linear size falling in a range from about 1 μm to about 500 μm, from 5 μm to about 100 μm, and/or about 5 μm to about 25 μm, which mimics individual cell size, may be formed in the ECM material 120. Molten material from the sacrificial microparticles 108 may diffuse away through the ECM material 120 over time. The sacrificial microparticles 108 may comprise gelatin (e.g., type A or B gelatin), chitosan, alginate, and/or gum arabic. In one example, the sacrificial microparticles 108 may comprise type A gelatin and chitosan. Material selection for the sacrificial microparticles 108 may depend on the bioprinting application. In the brain, for example, gelatin and collagen are not the primary components of the ECM, and thus a biologically inert microparticle such as alginate may be more desirable for modeling the brain microenvironment.

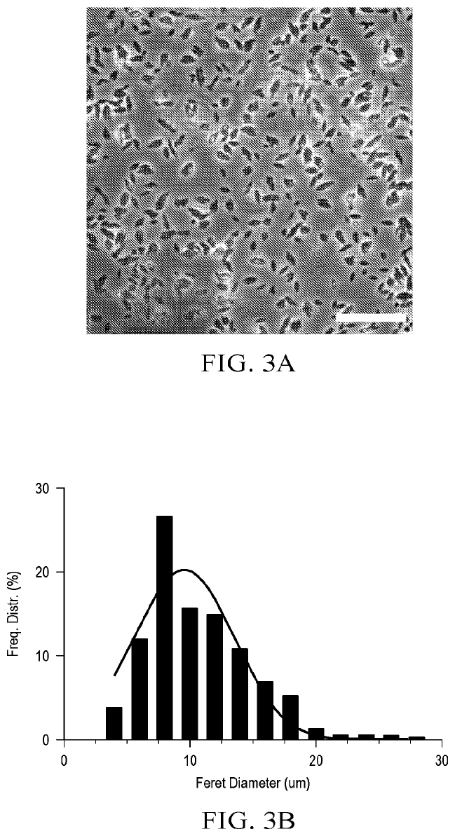

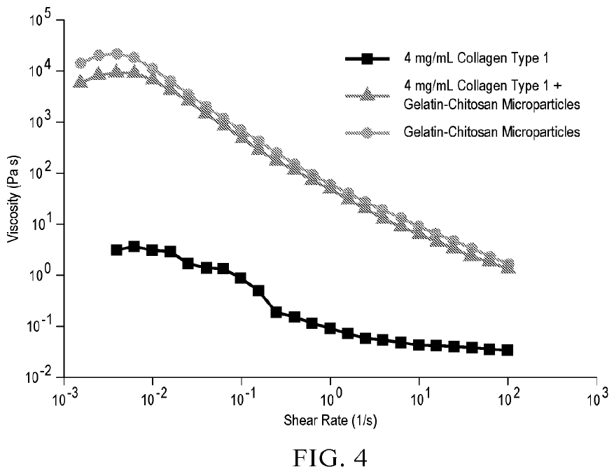

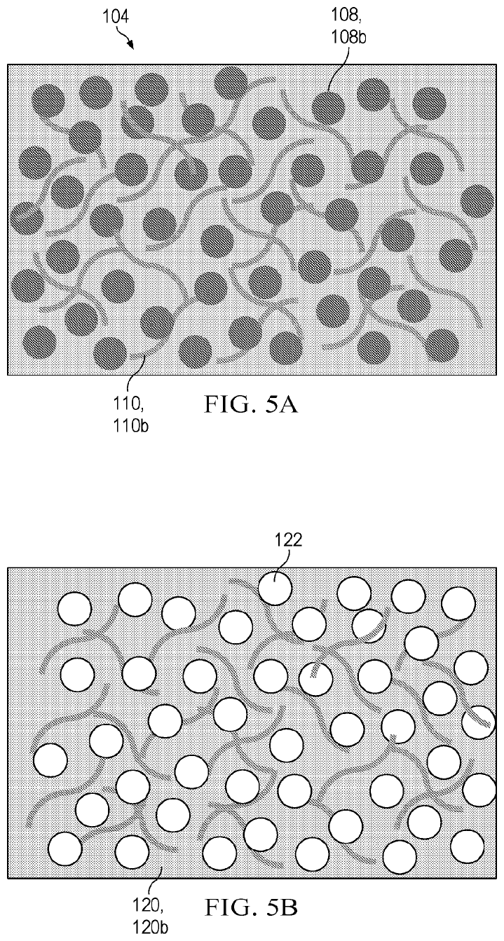

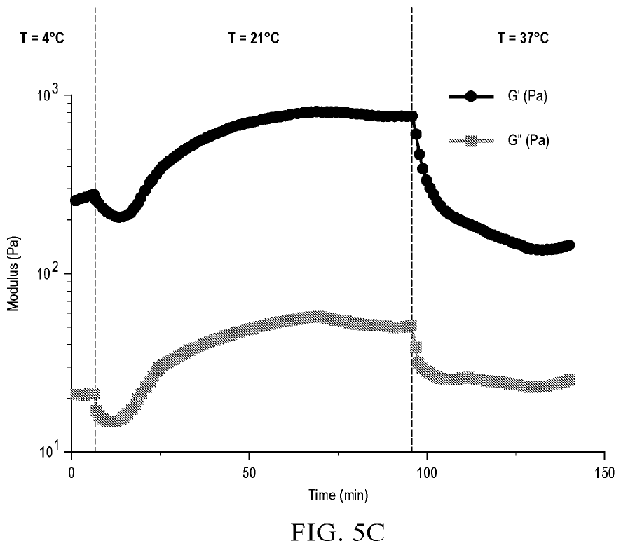

Sacrificial particles made of type A gelatin with chitosan and having a particle size (width or diameter) of approximately 10 μm have been prepared, as shown in FIGS. 3A and 3B. The sacrificial particles are designed to be similar in size to individual cells, which is important because this causes the cells to sense a three-dimensional environment as opposed to a curved two-dimensional surface. The sacrificial particles may be incorporated into an ECM precursor, such as uncrosslinked collagen type 1, to form a bioink. Collagen type 1 is an important example as it is the most abundant protein in the human body. FIG. 5 plots viscosity as a function of shear rate and reveals that the bioink exhibits a yield stress and is printable.

A method of fabricating a tissue/organ model or a therapeutic construct that may be implanted into a patient's body is described in reference to FIG. 1. The method may include extruding a bioink 106 comprising a first ECM precursor 110a and first sacrificial microparticles 108a through a nozzle 102 moving relative to a deposition bath 104. As explained above, the first sacrificial microparticles 108a may function as rheological modifiers in the bioink 106, enhancing or enabling the printability of the first ECM precursor 110a. The bioink 106 may further comprise one or more cell types, drugs, toxins, vaccines, proteins, and/or hormones, such as growth factors, growth inhibitors, cytokines, steroids and/or morphogens. The one or more cell types may include cells incorporated as individual cells, cell clusters/aggregates, and/or organoids. The cells may further be encapsulated (e.g., with an immunoprotective polymer gel). As the nozzle 102 moves, an extruded filament 112 comprising the bioink 106 is forced from the nozzle 102 and deposited into the bath 104. The nozzle 102 may be positioned above and/or within the bath 104 during the extrusion and may be moved along any desired two- or three-dimensional pathway. Referring now to FIG. 5A, the deposition bath 104 may comprise a second ECM precursor 110b, which may be the same as or different from the first ECM precursor 110a. Ideally there is a smooth interface between the second ECM material 110b of the deposition bath 104 and the first ECM material 110a of the extruded filament 112. In one example, the first and second ECM materials 110a, 110b may comprise collagen, and the first sacrificial microparticles 108a may comprise gelatin, or more specifically, gelatin and chitosan. As discussed below and as illustrated in FIG. 5A, the deposition bath may also comprise the sacrificial microparticles 108.

After deposition of the extruded filament 112 into the bath 104, the first ECM precursor 110a is crosslinked to form a first ECM material 120a, as illustrated in FIG. 2A, and the second ECM precursor 110b is crosslinked to form a second ECM material 120b, as illustrated in FIG. 5B, which again may be the same as or different from the first ECM material 120a. In the former case, a seamless matrix comprising the ECM material 120a may be formed over the entire region originally occupied by the deposition bath 104 and the extruded filament 112. In the latter case, the first ECM material 120a may extend over the region(s) (e.g., the print path) originally occupied by the extruded filament 112 and may effectively be encapsulated by the second ECM material 120b. The crosslinking may be effected by an increase in temperature, light exposure, and/or a chemical agent. The impact of crosslinking is to transform the gel-like bioink 106 or deposition bath 104 into a stable polymer network. Each of the first and second ECM materials 120a, 120b may comprise a natural or synthetic polymer as described above, and each of the first and second ECM precursors 110a, 110b may comprise an uncrosslinked polymer, that is, the respective natural or synthetic polymer prior to crosslinking. As indicated above, crosslinking may be induced by heat, light, an enzyme and/or a chemical agent, but in each scenario the crosslinking temperature of the uncrosslinked polymer may be understood to refer to the temperature at which the crosslinking reaction takes place. To ensure crosslinking does not occur prematurely, the extrusion and deposition may take place at a reduced temperature, e.g., at a temperature below the crosslinking temperature of the natural or synthetic polymer. For example, for a first and/or second ECM precursor 110a, 110b comprising collagen, extrusion and deposition may take place at a temperature in a range from 1-10° C., followed by crosslinking at a temperature in a range from 15-25° C.

After the crosslinking, the first sacrificial microparticles 108a are melted, that is, heated at or above a melting temperature thereof, to form pores 122 in the first ECM material 120a, as illustrated in FIG. 2B. The pores 122 preferably have a linear size (width and/or diameter) that mimics individual cell size, such as in a range from about 5 μm to about 100 μm, or in the range from about 5 μm to about 25 μm. The melting temperature of the first sacrificial microparticles 108a is above the crosslinking temperature of at least the first ECM material 120a and preferably above the crosslinking temperature(s) of both the first and second ECM materials 120a, 120b, which may be the same or different, as indicated above. In an example where the first sacrificial microparticles 108a comprise gelatin, or gelatin and chitosan, the melting temperature may lie in a range from about 32-42° C.

FIG. 5C shows modulus as a function of time for a bioink 106 comprising uncrosslinked collagen with gelatin-chitosan microparticles exposed to a temperature of 4° C., 21° C. and then 37° C. At 4° C., uncrosslinked collagen with gelatin-chitosan microparticles exhibits a yield stress and behaves like a solid; increasing the temperature to 21° C. induces collagen crosslinking; and further increasing the temperature to 37° C. melts the gelatin-chitosan microparticles, but leaves behind a stable collagen network.

Like the bioink 106, the deposition bath 104 may further include sacrificial microparticles 108 (“second sacrificial microparticles 108b”). In this case, the second sacrificial microparticles 108b may or may not be employed for rheology control, in contrast to the first sacrificial microparticles 108a present in the bioink 106, which is extruded through the nozzle 102. The second sacrificial microparticles 108b may function primarily as a source of controlled porosity in the second ECM material 120b. Accordingly, the method may further comprise, after crosslinking the second ECM precursor 110b, melting the second sacrificial microparticles 108b to form pores 122 in the second ECM material 120b. The second sacrificial microparticles 108b melt at a temperature above the crosslinking temperature of at least the second ECM material 120b and preferably above the crosslinking temperature(s) of both the first and second ECM materials 120a, 120b. The first and/or second sacrificial microparticles 108a, 108b may have any of the characteristics set forth above. Molten material from the sacrificial microparticles 108a, 108b may diffuse away through the ECM material(s) 120a, 120b over time. The deposition bath 104 may also or alternatively include one or more cell types, drugs, toxins, vaccines, proteins, and/or hormones, such as growth factors, growth inhibitors, cytokines, steroids and/or morphogens.

In some applications, it may be advantageous to replace the pores 122 formed by the melting of the first and/or second sacrificial microparticles 108a, 108b with what may be described as a reinforcement phase by crosslinking the molten material, preferably shortly after melting, before the molten material has time to diffuse from the pores 122. In such a situation, after melting the first and/or second sacrificial microparticles 108a, 108b, molten material may be exposed to light, chemical agent, and/or enzyme to induce crosslinking, thereby filling the pores 122 with a crosslinked polymer.

The organ/tissue model or therapeutic construct may further be vascularized. Accordingly, the method may also include, prior to crosslinking the first and/or second ECM precursors 110a, 110b, extruding a sacrificial ink through the nozzle 102 and depositing an extruded filament comprising the sacrificial ink into the deposition bath 104 as the nozzle 102 moves along a predetermined print path. The sacrificial ink may comprise, for example, gelatin (e.g., type A or B gelatin), chitosan, alginate, and/or gum arabic. The sacrificial ink may be formed from the first and/or second sacrificial microparticles, in one example. After deposition of an extruded filament 112 comprising the sacrificial ink, and after crosslinking the first and/or second ECM precursors 110a, 110b, the sacrificial ink may be melted to form a vascular channel extending along the print path through the first and/or second ECM materials 120a, 120b. The vascular channel may have a size and shape determined by the extruded filament 112. Advantageously, the sacrificial ink may melt at a temperature above the crosslinking temperature(s) of the first and/or second ECM materials 120a, 120b. A suspension of endothelial cells may be injected into the vascular channel after melting the sacrificial ink, and/or endothelial cells may be incorporated into the sacrificial ink prior to extrusion through the nozzle 102, thereby promoting endothelialization of the vascular channel.

The one or more cell types that may be included in the bioink, the deposition bath, the vascular channel, and/or otherwise introduced into the organ/tissue model or the therapeutic construct formed according to any example in this disclosure, may comprise any mammalian cell type selected from cells that make up the mammalian body, including germ cells, somatic cells, and stem cells. The term “germ cells” refers to any line of cells that give rise to gametes (eggs and sperm). The term “somatic cells” refers to any biological cells forming the body of a multicellular organism; any cell other than a gamete, germ cell, gametocyte or undifferentiated stem cell. Examples of somatic cells include fibroblasts, chondrocytes, osteoblasts, tendon cells, mast cells, wandering cells, immune cells, pericytes, inflammatory cells, endothelial cells, myocytes (cardiac, skeletal and smooth muscle cells), adipocytes (i.e., lipocytes or fat cells), parenchyma cells (neurons and glial cells, nephron cells, hepatocytes, pancreatic cells, lung parenchyma cells) and non-parenchymal cells (e.g., sinusoidal hepatic endothelial cells, Kupffer cells and hepatic stellate cells). The term “stem cells” refers to cells that have the ability to divide for indefinite periods and to give rise to virtually all of the tissues of the mammalian body, including specialized cells. The stem cells include pluripotent cells, which upon undergoing further specialization become multipotent progenitor cells that can give rise to functional or somatic cells. Examples of stem and progenitor cells include hematopoietic stem cells (adult stem cells; i.e., hemocytoblasts) from the bone marrow that give rise to red blood cells, white blood cells, and platelets; mesenchymal stem cells (adult stem cells) from the bone marrow that give rise to stromal cells, fat cells, and types of bone cells; epithelial stem cells (progenitor cells) that give rise to the various types of skin cells; neural stem cells and neural progenitor cells that give rise to neuronal and glial cells; and muscle satellite cells (progenitor cells) that contribute to differentiated muscle tissue. The cells may also or alternatively comprise tumor or cancer cells, such as carcinoma, sarcoma, leukemia, lymphoma, melanoma, and/or multiple myeloma cells.

As an alternative printing approach to the bioink printing method described above, where the bioink includes an ECM precursor along with sacrificial microparticles to promote printability, the method may comprise printing a bioink into a deposition bath that includes an ECM precursor and sacrificial microparticles. In this alternative approach, the bioink may include a suspension of cells, for example, but may be devoid of an ECM precursor and/or sacrificial microparticles.

Accordingly, the method of fabricating a tissue/organ model or therapeutic construct may include extruding a bioink through a nozzle moving relative to a deposition bath, where in this example the deposition bath comprises a first extracellular matrix (ECM) precursor and first sacrificial microparticles. The deposition bath may also include one or more cell types, drugs, toxins, vaccines, proteins, and/or hormones, such as growth factors, growth inhibitors, cytokines, steroids and/or morphogens. The one or more cell types may include cells incorporated as individual cells, cell clusters/aggregates, and/or organoids. The cells may further be encapsulated (e.g., with an immunoprotective polymer gel). As the nozzle moves, an extruded filament comprising the bioink is deposited into the deposition bath. After the depositing, the first ECM precursor is crosslinked to form a first ECM material. To ensure that crosslinking does not occur prematurely, the extrusion and deposition may take place at a reduced temperature, e.g., at a temperature below the crosslinking temperature of the natural or synthetic polymer. After the crosslinking, the first sacrificial microparticles are melted to form pores in the first ECM material. The pores preferably have a linear size (width and/or diameter) that mimics individual cell size, such as in a range from about 5 μm to about 100 μm, or in the range from about 5 μm to about 25 μm.

The bioink may comprise one or more cell types, drugs, toxins, vaccines, proteins, and/or hormones, such as growth factors, growth inhibitors, cytokines, steroids and/or morphogens. The bioink may also or alternatively comprise an ECM precursor (a second ECM precursor), in which case the method may further comprise, after the depositing, crosslinking the second ECM precursor to form a second ECM material. The first and second ECM materials may be the same or different, as in the method described above. The first and second ECM materials may comprise a natural or synthetic polymer selected from, for example, collagen, fibrinogen, reconstituted extracellular matrices, modified matrix-derived proteins (e.g., gelatin), modified glycosaminoglycans (e.g., hyaluronic acid, chondroitin sulfate), modified polysaccharides (e.g., alginate, dextran, chitosan), and functionalized polyethylene glycol. These polymers may be modified/functionalized with photocrosslinkable moieties like methacrylate, acrylate, norbornene, and/or allyl groups. Alternatively, they may be modified/functionalized with dynamic covalent crosslinkers (e.g., imine and acylhydrazone) and/or host-guest interactions (e.g., adamantane and beta-cyclodextrin), and the first and second ECM precursors may comprise an uncrosslinked polymer, that is, the respective natural or synthetic polymer prior to crosslinking. Crosslinking may be induced by heat, light, an enzyme and/or a chemical agent, but in each scenario the crosslinking temperature of the uncrosslinked polymer may be understood to refer to the temperature at which the crosslinking reaction takes place. The bioink may also or alternatively comprise second sacrificial microparticles, in which case the method may comprise, after the crosslinking of the second ECM precursor, melting the second sacrificial microparticles to form pores in the second ECM material. As above, the first and/or second sacrificial microparticles may comprise gelatin, chitosan, alginate, and/or gum arabic.

In some applications, it may be advantageous to replace the pores formed by the melting of the first and/or second sacrificial microparticles with what may be described as a reinforcement phase by crosslinking the molten material, preferably shortly after melting, before the molten material has time to diffuse from the pores. In such a situation, after melting the first and/or second sacrificial microparticles, molten material may be exposed to light, chemical agent, and/or enzyme to induce crosslinking, thereby filling the pores with a crosslinked polymer.

The organ/tissue model or therapeutic construct may further be vascularized. Accordingly, the method may also include, prior to crosslinking the first and/or second ECM precursors, extruding a sacrificial ink through the nozzle and depositing an extruded filament comprising the sacrificial ink into the deposition bath as the nozzle moves along a predetermined print path. The sacrificial ink may comprise, for example, gelatin (e.g., type A or B gelatin), chitosan, alginate, and/or gum arabic. The sacrificial ink may be formed from the first and/or second sacrificial microparticles, in one example. After deposition of an extruded filament comprising the sacrificial ink, and after crosslinking the first and/or second ECM precursors, the sacrificial ink may be melted to form a vascular channel extending along the print path through the first and/or second ECM materials. The vascular channel may have a size and shape determined by the extruded filament. Advantageously, the sacrificial ink may melt at a temperature above the crosslinking temperature(s) of the first and/or second ECM materials. A suspension of endothelial cells may be injected into the vascular channel after melting the sacrificial ink, and/or endothelial cells may be incorporated into the sacrificial ink prior to extrusion through the nozzle, thereby promoting endothelialization of the vascular channel. Once these vascular channels (or macro-vessels) are formed, capillary vessels (or micro-vessels) may form via self-assembly.

As indicated above, the bioink employed in this alternative method may include one or more cell types but may be devoid of an ECM precursor and/or sacrificial microparticles. The one or more cell types may include cells incorporated as individual cells, cell clusters/aggregates, and/or organoids. The cells may further be encapsulated (e.g., with an immunoprotective polymer gel). In such an example, bioink may further include a liquid carrier for the cells, such as a saline solution. For example, a suspension or slurry of cells may be printed into a deposition bath that includes an ECM precursor such as collagen and optionally sacrificial microparticles.

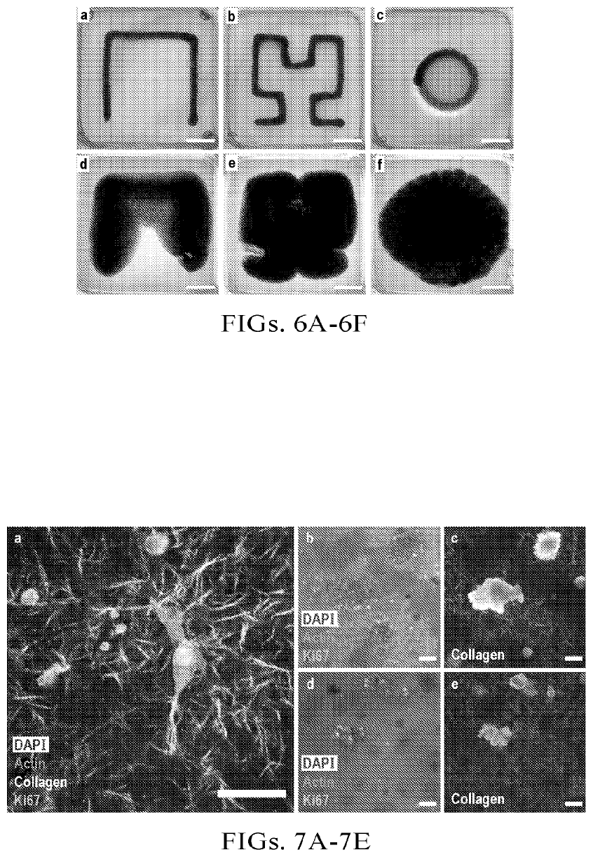

FIGS. 6A-6F illustrate the improvements in printing resolution and shape retention when a cellular bioink is extruded through a nozzle and deposited into a bath including collagen and microparticles, in comparison with deposition into a bath including just collagen. In this experimental example, the cellular bioink includes B16-F10 melanoma cells (approximately 250 million cells/mL) in phosphate-buffered saline, and the cellular bioink is printed into either (FIGS. 6A-6C) a deposition bath of collagen and gelatin-chitosan microparticles or (FIGS. 6D-6F) collagen alone. The images show (6A, 6D) a first-order Hilbert curve, (6B, 6E) second-order Hilbert curve, (6C-6F) and helix with 4 mm diameter and 2 mm pitch (scale bar=2 mm). Importantly, the presence of the microparticles in the deposition bath appears to enable filaments of ˜550 μm in diameter to be printed and to retain their shape.

Notably, the approach may yield a fibrillar architecture, which is an important feature of collagen in vivo. In addition, cells embedded within the printable collagen type 1 material exhibit key cellular processes such as proliferation and migration. Immunofluorescent imaging of B16-F10 cells printed within the collagen/microparticle bath reveal that the cells are able to spread within the matrix and are proliferative, as evidenced by the Ki67 proliferation marker. In addition, confocal reflectance imaging shows that the collagen within the collagen/microparticle bath forms a fibrillar network, as indicated in FIG. 7A. FIGS. 7B and 7C show that B16-F10 melanoma cells also formed proliferative aggregates within the collagen/microparticle bath. FIGS. 7D and 7E show B16-F10 melanoma cells embedded within 2 mg/mL collagen as a control. The scale bars are equivalent to 50 μm.

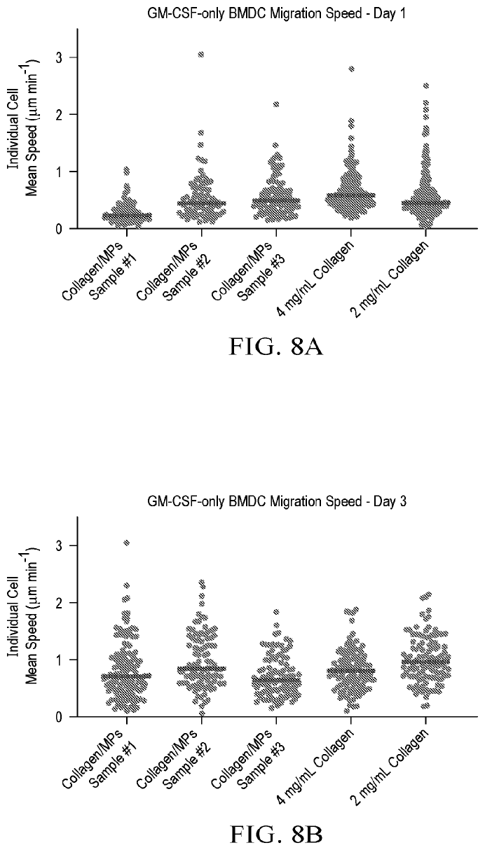

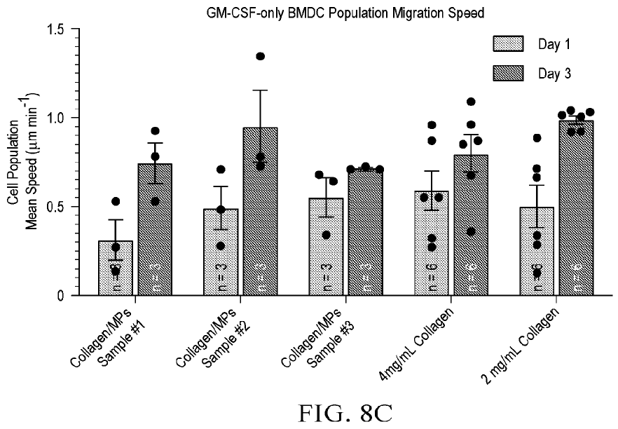

FIGS. 8A-8C show results from a migration assay of bone marrow-derived dendritic cells (BMDCs) embedded within a collagen/microparticle bioink or bath. The migration of primary BMDCs embedded within the collagen/microparticles was assessed on (FIG. 8A) day 1 and (FIG. 8B) day 3. Collagen/MP samples #1-3 represent independent mixtures of collagen and microparticles and show that the approach is reproducible. Referring to FIG. 8C, the population migration speed, in which each data point is the mean cell speed within an independent gel, shows that the migration speed within the collagen/microparticle bioink is not statistically different from the collagen controls for both day 1 and day 3.

Generally speaking, the tissue/organ model fabricated as described above may be a tumor or cancer model used to study the development and progression of cancer and/or to test new treatments. In such an example, the bioink employed in the method may comprise tumor cells, such as the melanoma cells used in the example of FIGS. 6A-6E, and the deposition bath may include immune cells (e.g., from a patient or allogenic). Alternatively, the deposition bath may not include immune cells, and instead the method may include exposing the tissue/organ model to immune cells after the tissue/organ model is constructed. In one example, encapsulated T cells are dispersed in a deposition bath including an ECM precursor and sacrificial microparticles, and a bioink comprising melanoma cells is extruded through a nozzle to deposit a filament comprising the melanoma cells into the deposition bath. After crosslinking the ECM precursor and melting the sacrificial microparticles, the tumor model is formed. Experiments have demonstrated that the T cells can migrate through the ECM material (e.g., collagen) and remain functional, as demonstrated by T cell-mediated killing of the tumor (melanoma) cells within the matrix material.

It is noted that the bioinks and the ECM material become more transparent after removal (melting) of the sacrificial microparticles. Accordingly, the tissue/organ model may be exposed to light for photo-patterning or photo-ablation. For example, it is contemplated that drugs, toxins, vaccines, proteins, and/or hormones, such as growth factors, growth inhibitors, cytokines, steroids and/or morphogens, may be photo-patterned within the bioink and/or ECM material after removal of the sacrificial microparticles. Also or alternatively, light-based chemistry may be employed to spatially pattern mechanical differences in the tissue/organ model or therapeutic construct. In one example, a subsequent crosslinking reaction, that is, a crosslinking reaction that follows melting of the sacrificial microparticles, may be photo-activated for selective stiffening of the ECM material. It is also contemplated that photo-ablation may be employed after removal of the sacrificial microparticles to create filaments or other features having finer dimensions than possible with extrusion-based printing.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein.

Furthermore, the advantages described above are not necessarily the only advantages of the invention, and it is not necessarily expected that all of the described advantages will be achieved with every embodiment of the invention.

1. A bioink for extrusion-based printing, the bioink comprising:
an extracellular matrix (ECM) precursor comprising an uncrosslinked polymer; and
sacrificial microparticles dispersed in the ECM precursor,
wherein the sacrificial microparticles have a melting temperature above a crosslinking temperature of the uncrosslinked polymer.

2. The bioink of claim 1, wherein the sacrificial microparticles have a width or diameter of about 1 μm to about 100 μm.

3. The bioink of claim 1, wherein the sacrificial microparticles comprise gelatin, chitosan, alginate, and/or gum arabic.

4-5. (canceled)

6. The bioink of claim 1, further comprising one or more cell types, drugs, toxins, vaccines, proteins, and/or hormones, such as growth factors, growth inhibitors, cytokines, steroids and/or morphogens.

7. The bioink of claim 6, wherein the one or more cell types comprise single cells, cell clusters/aggregates, and/or organoids, and/or
wherein the one or more cell types are encapsulated within an immunoprotective polymeric hydrogel.

8-14. (canceled)

15. A method of fabricating a tissue/organ model or therapeutic construct, the method comprising:
extruding a bioink comprising a first extracellular matrix (ECM) precursor and first sacrificial microparticles through a nozzle moving relative to a deposition bath;
depositing an extruded filament comprising the bioink into the deposition bath as the nozzle moves;
after the depositing, crosslinking the first ECM precursor to form a first ECM material; and
after the crosslinking, melting the first sacrificial microparticles to form pores in the first ECM material, thereby forming a tissue/organ model or therapeutic construct.

16-17. (canceled)

18. The method of claim 15, wherein the bioink further comprises one or more cell types, drugs, toxins, vaccines, proteins, and/or hormones, such as growth factors, growth inhibitors, cytokines, steroids and/or morphogens.

19. The method of claim 18, wherein the one or more cell types include single cells, cell clusters/aggregates, and/or organoids, and/or
wherein the one or more cell types are encapsulated within an immunoprotective polymeric hydrogel.

20. The method of claim 15, wherein the deposition bath comprises a second extracellular matrix (ECM) precursor, and further comprising, after the depositing, and crosslinking the second ECM precursor to form a second ECM material.

21. The method of 20, wherein the first and second ECM materials are the same.

22. The method of claim 20, wherein the first and second ECM materials are different.

23. The method of claim 15, wherein the deposition bath further comprises one or more cell types, drugs, toxins, vaccines, proteins, and/or hormones, such as growth factors, growth inhibitors, cytokines, steroids and/or morphogens.

24. The method of claim 15, wherein the one or more cell types include single cells, cell clusters/aggregates, and/or organoids, and/or
wherein the one or more cell types are encapsulated within an immunoprotective polymeric hydrogel.

25. The method of claim 15, wherein the deposition bath further comprises second sacrificial microparticles, and further comprising, after the crosslinking of the second ECM precursor, melting the second sacrificial microparticles to form pores in the second ECM material.

26-27. (canceled)

28. The method of claim 15, further comprising, after the melting of the first and/or second sacrificial microparticles, exposing molten material from the first and/or second sacrificial microparticles to light, chemical agent, and/or enzyme to induce crosslinking, thereby filling the pores with a crosslinked polymer.

29. The method of claim 15, wherein the first and/or second ECM materials comprise a natural or synthetic polymer selected from the group consisting of: collagen, fibrinogen, reconstituted extracellular matrices, modified matrix-derived proteins (e.g., gelatin), modified glycosaminoglycans (e.g., hyaluronic acid, chondroitin sulfate), modified polysaccharides (e.g., alginate, dextran, chitosan), and functionalized polyethylene glycol, and
wherein the first and/or second ECM precursors comprise the natural or synthetic polymer prior to crosslinking.

30. The method of claim 29, wherein the natural or synthetic polymer is functionalized with
one or more photocrosslinkable moieties comprising methacrylate, acrylate, norbornene, and/or allyl groups,
one or more dynamic covalent crosslinkers comprising imine and/or acylhydrazone, and/or
one or more host-guest interactions comprising adamantane and/or beta-cyclodextrin.

31. The method of claim 15, wherein the first and/or second sacrificial microparticles comprise gelatin, chitosan, alginate, and/or gum arabic.

32. The method of claim 15, further comprising, prior to the crosslinking of the first and/or second ECM precursors, extruding a sacrificial ink through the nozzle and depositing an extruded filament comprising the sacrificial ink into the deposition bath as the nozzle moves along a predetermined print path; and
after depositing the extruded filament comprising the sacrificial ink and after crosslinking the first and/or second ECM precursors, melting the sacrificial ink to form a vascular channel in the first and/or second ECM materials.

33. A method of fabricating a tissue/organ model or therapeutic construct, the method comprising:
extruding a bioink through a nozzle moving relative to a deposition bath, the deposition bath comprising a first extracellular matrix (ECM) precursor and first sacrificial microparticles;
depositing an extruded filament comprising the bioink into the deposition bath as the nozzle moves;
after the depositing, crosslinking the first ECM precursor to form a first ECM material; and
after the crosslinking, melting the first sacrificial microparticles to form pores in the first ECM material, thereby forming a tissue/organ model or therapeutic construct.

34-52. (canceled)
